(19) United States
(12) Patent Application Publication
(10) Pub. No.: US 2015/0190383 A1
(43) Pub. Date: Jul. 9, 2015

(54) HAIR CARE COMPOSITION WITH ANALGESIC

(71) Applicants: AIDA KALLEY, Gainesville, VA (US); LETTY CINTRON-PATTERSON, Bristow, VA (US)

(72) Inventors: AIDA KALLEY, Gainesville, VA (US); LETTY CINTRON-PATTERSON, Bristow, VA (US)

(21) Appl. No.: 14/150,135

(22) Filed: Jan. 8, 2014

(51) Int. Cl. A61K 31/445; A61Q 5/00; A61Q 5/12; A61K 8/97; A61K 8/92

(52) U.S. Cl. CPC A61K 31/445; A61K 8/97; A61K 8/922; A61Q 5/12; A61Q 5/00

(57) ABSTRACT

A hair detangling composition includes a conditioning material, a lubricant, a scent, and an anesthetic.

### BACKGROUND OF THE INVENTION

1. Field of the Invention

The present invention relates to hair care compositions used in assisting in the detangling of hair. More particularly, the invention relates to the incorporation of an anesthetic with the detangling composition to assist in the alleviation of pain that might be caused as one treats hair to remove tangles.

2. Description of the Related Art

As parents appreciate, children commonly have fine hair that is highly susceptible to tangling. Still further, children are often not diligent in combing their hair out, therefore, the problem of tangles is further exacerbated. When it is time to detangle a child's hair, the process can often result in fidgeting, discomfort, tears and even pain.

With this in mind, the present invention provides a composition to assist in detangling hair while simultaneously applying an anesthetic that minimizes any pain associated with the detangling process.

### SUMMARY OF THE INVENTION

It is, therefore, an object of the present invention to provide a hair detangling composition including a conditioning material, a lubricant, a scent, and an anesthetic.

It is also an object of the present invention to provide a hair detangling composition wherein the anesthetic is bupivacaine.

It is another object of the present invention to provide a hair detangling composition wherein the anesthetic is bupivacaine 0.5%.

It is a further object of the present invention to provide a hair detangling composition wherein the lubricant includes boiled flax seed water.

It is also an object of the present invention to provide a hair detangling composition wherein the conditioning material includes aloe vera, glycerin, shea butter, avocado oil, coconut oil and olive oil.

It is another object of the present invention to provide a hair detangling composition wherein the scent includes orange extract.

Other objects and advantages of the present invention will become apparent from the following detailed description when viewed in conjunction with the accompanying drawings, which set forth certain embodiments of the invention.

### DESCRIPTION OF THE PREFERRED EMBODIMENT

The detailed embodiment of the present invention is disclosed herein. It should be understood, however, that the disclosed embodiment is merely exemplary of the invention, which may be embodied in various forms. Therefore, the details disclosed herein are not to be interpreted as limiting, but merely as a basis for teaching one skilled in the art how to make and/or use the invention.

As discussed above, the present invention relates to the incorporation of an anesthetic with hair care products, a composition for the purpose of assisting in the alleviation of pain that might be caused as one treats hair to remove tangles.

Briefly, the detangling composition of the present invention includes a conditioning material(s), a lubricant(s), a scent(s), and an anesthetic(s). This combination of conditioning materials, lubricants, scents, and an anesthetic results in a highly effective tool for parents attempting to detangle their children's hair. Briefly, and in accordance with a preferred embodiment of the present invention, detangling composition is composed of aloe vera, glycerin, shea butter, avocado oil, coconut oil, olive oil, essential oil, boiled flax seed water and orange extract in the composition of a gel, cream or liquid depending on the texture of the hair or results desired. In addition to these base components the anesthetic bupivicaine is incorporated into the composition. In accordance with a preferred embodiment, the bupivacaine is incorporated as bupivicaine 0.5% (which is appreciated to be a composition containing 5 mg/ml). Although bupivacaine is disclosed in accordance with a preferred embodiment of the present invention, it is appreciated other anesthetic compositions may be used within the spirit of the present invention.

With an understanding of the various components making up the present composition, it is appreciated that the boiled flax seed water provides a lubricating composition assisting in the detangling of hair. In addition, the aloe vera, glycerin, shea butter, avocado oil, coconut oil and olive oil provide the conditioning component for the present composition. The orange extract, or scent agent, provides a scent for the present composition. Finally, the bupivacaine or comparable agent provides an anesthetic effect that enhances the detangling process by minimizing any pain that might be associated with the pulling of hair as it is detangled.

More specifically, the solution is approximately 1.74% aloe vera by volume; approximately 1.74% shea butter by volume; approximately 1.74% avocado oil by volume; approximately 3.48% coconut oil by volume; approximately 6.97% olive oil by volume; and approximately 1.74% boiled flax seed water by volume. The composition additionally includes approximately 0.70% bupivacaine 0.5% (or comparable anesthetic agent ranging up to 2%) by volume, as well as less than 1% essential oil by volume, less than 1% glycerin by volume, and less than 1% orange extract by volume The remaining volume of the composition is distilled water.

In accordance with one embodiment, the composition is formulated in the following specific volumes:

aloe vera—5 ml;

glycerin—5 drops;

shea butter—5 ml;

avocado oil—5 ml;

coconut oil—10 ml;

olive oil—20 ml;

essential oil—10 drops;

distilled water—235 ml;

bupivicaine 0.5% —1 ml—10 ml (which constitutes 5-50 mg bupivicaine);

boiled flax seed water—5 ml up to 10 ml; and

orange extract—10 drops or comparable scent agent.

While the preferred embodiments have been shown and described, it will be understood that there is no intent to limit the invention by such disclosure, but rather, is intended to cover all modifications and alternate constructions falling within the spirit and scope of the invention.

1. A hair detangling composition, comprising:
a conditioning material,
a lubricant,
a scent, and
an anesthetic.

2. The hair detangling composition according to claim 1, wherein the anesthetic is bupivacaine.

3. The hair detangling composition according to claim 2, wherein the anesthetic is bupivacaine 0.5%.

4. The hair detangling composition according to claim 3, wherein the lubricant includes boiled flax seed water.

5. The hair detangling composition according to claim 4, wherein the conditioning material includes aloe vera, glycerin, shea butter, avocado oil, coconut oil and olive oil.

6. The hair detangling composition according to claim 5, wherein the scent includes orange extract.

7. The hair detangling composition according to claim 1, wherein the lubricant includes boiled flax seed water.

8. The hair detangling composition according to claim 7, wherein the conditioning material includes aloe vera, glycerin, shea butter, avocado oil, coconut oil and olive oil.

9. The hair detangling composition according to claim 8, wherein the scent includes orange extract.

10. The hair detangling composition according to claim 1, wherein the conditioning material includes aloe vera, glycerin, shea butter, avocado oil, coconut oil and olive oil.

11. The hair detangling composition according to claim 1, wherein the scent includes orange extract.
